(19) United States
(12) Patent Application Publication
(10) Pub. No.: US 2006/0122605 A1
(43) Pub. Date: Jun. 8, 2006

(54) Translational plate with cover blocking system

(76) Inventors: Sean S. Suh, Plymouth Meeting, PA (US); David S. Rathbun, Gap, PA (US)

Correspondence Address: JONES DAY, 222 EAST 41ST ST, NEW YORK, NY, 10017, US

(21) Appl. No.: 11/006,113

(22) Filed: Dec. 6, 2004

(51) Int. Cl. A61F 2/30

(52) U.S. Cl. 606/69

(57) ABSTRACT

An apparatus includes a bone fastener, a bone fixation device and a bone fastener retainer device. The head of the fastener has a convex lower surface with a first arcuate contour, and has a convex upper surface with a second arcuate contour. The fixation device is configured to receive the fastener in an installed position in which the convex lower surface of the fastener head rests on a concave surface of the bone fixation device that also has the first arcuate contour. Additionally, the retainer device is configured to be mounted on the fixation device in a position in which a concave surface of the retainer device overlies the convex upper surface of the fastener head. The concave surface of the retainer device also has the second arcuate contour. The bone fastener retainer device may be receivable over the fixation device in a mechanical interlock with the fixation device.

### FIELD OF THE INVENTION

This technology relates to bone screws for attaching fixation devices to bones, and particularly relates to devices that retain the bone screws in their installed positions.

### BACKGROUND OF THE INVENTION

Orthopedic fixation devices such as plates are frequently coupled to bone with fasteners inserted through plate holes. It is known that securing such fasteners to the bone plate, for example through the use of expansion-head screws, can decrease the incidence of loosening of the fixation assembly post-operatively. It is also known that a bushing may be disposed in each plate hole to receive the fastener to permit polyaxial movement so that the fastener may be angulated at a surgeon-selected angle. However, polyaxial movement of fasteners through set plate hole locations only increases attachment alternatives of the fasteners themselves. The plate holes remain fixed in relation to each other and to the longitudinal axis of the plate.

Typically, a spinal fixation plate is applied to the anterior side of the affected vertebrae to span at least one affected disc space or vertebra (i.e. one in which at least a portion of the disc has been removed and a spinal fusion spacer has been inserted). The plate is fixed to the vertebrae using bone screws and acts to keep the vertebrae generally aligned during the initial period following fixation in which fusion of the spacer to the adjacent vertebrae occurs. The plate also may act to prevent the spacer from being expelled from the disc space during this initial period.

Where a spinal fusion spacer is implanted between a pair of vertebrae to be fused, the spacer rests on the endplates of the vertebrae. The outer circumference of the end plates comprises hard cortical bone and thus provides the best surface upon which to seat the spacer. The center portion of the endplates comprises a thin cortical bone shell overlying a core of softer cancellous bone. Most, if not all, of the spacer contact surface, however, may be located in this center portion.

Subsequent to placement of the spacer, the surgeon typically compresses the disc space by pressing the adjacent vertebrae together. This compression ensures a good engagement between the spacer and the endplates, increasing the chances that fusion will occur. Often in the period immediately following surgery, the spacer may subside slightly into the under-portion of the endplates, or the space between the vertebral endplates may decrease due to graft resorption (in the case of allograft spacers).

Where a rigid fixation plate is used to connect the vertebrae, this subsidence may tend to shift more of the spinal load to the plate than is desirable. Such load shifting can also occur due to inaccuracies in installing the plate to the vertebrae. In extreme circumstances, this load shifting can result in non-fusion of the spacer to the vertebra, since firm compression between the spacer and the vertebrae is one factor contributing to successful fusion.

Accordingly, there exists a need for a fixation system which provides the desired support to the vertebrae to be fused, and which allows limited translation of the vertebrae with respect to at least a portion of the plate, thereby limiting the undesirable effects of load shielding by the plate due to graft subsidence caused by settling or normal forces experienced in the spinal column. Promoting fusion of the adjacent vertebrae may thus accomplished.

Translation plates which compensate for this subsidence by providing the aforementioned benefits of a rigid fixation plate (general vertebral alignment, and prevention of spacer expulsion), while allowing at least one vertebra to move with respect to the plate to compensate for post-surgical subsidence, may be desirable. This compensation may permit the majority of the spinal column load to be borne by the spacer rather than the plate.

Therefore, there exists a need for a fastener retaining device that can be coupled to a translational plate for preventing screw back-out. There also exists a need for such a retainer device to be conveniently situated in or around the plate, so as not to interfere with the insertion and/or placement of fasteners. There further exists a need for a retainer device to be bendable and/or shiftable by a surgeon without the use of strenuous force.

### SUMMARY OF THE INVENTION

An apparatus may include a bone fastener, a bone fixation device and a bone fastener retainer device. The head of the fastener may have a convex lower surface with a first arcuate contour, and has a convex upper surface with a second arcuate contour. The fixation device may be configured to receive the bone fastener in an installed position in which the convex lower surface of the fastener head rests on a concave surface of the fixation device that also has the first arcuate contour. Additionally, the retainer device may be configured to be mounted on the fixation device in a position in which a concave surface of the retainer device overlies the convex upper surface of the fastener head. The concave surface of the retainer device also may have the second arcuate contour.

Summarized differently, an apparatus may include a bone fixation device with a plurality of bone fastener openings, and further may include a bone fastener retainer device that may be receivable over the fixation device in a mechanical interlock with the fixation device. It may be preferable for the fixation device to have opposed edge surfaces that are spaced apart from each other a first distance. The retainer device may have an unstressed condition in which opposite edge surfaces of the retainer device are spaced apart from each other a second distance that is greater than the first distance. The retainer device may be elastically deflectable from the unstressed condition to a bowed condition. When the retainer device is in the bowed condition, the opposite edge surfaces of the retainer device may be spaced apart from each other a third distance that is less than the first distance. This may enable the retainer device to be placed between the opposed edge surfaces of the fixation device when in the bowed condition, and to move into a tongue-and-groove fit with the fixation device by rebounding from the bowed condition.

A fixation assembly is described comprising: a bone fastener having a head, the head having a convex lower surface with a first arcuate contour and a convex upper surface with a second arcuate contour; a fixation device configured to receive the bone fastener in an installed position, wherein the convex lower surface of the fastener head rests on a concave surface of the fixation device also substantially having the first arcuate contour; and a bone fastener retainer device configured to be mounted on the fixation device in an installed position, wherein a concave surface of the bone fastener retainer device overlies the convex upper surface of the head, wherein the concave surface of the bone fastener retainer device also substantially has the second arcuate contour.

The first and second arcuate contours may be substantially spherical. The first and second arcuate contours may have substantially equal radii of curvature.

The fixation device may be a plate, and wherein the concave surface of the fixation device is an inner edge surface within an opening through the plate. The opening may be substantially slot-shaped, and capable of receiving at least a portion of a bone fastener, the opening further having a longitudinal axis.

A bone fastener may be allowed to translate within the opening along the longitudinal axis. A bone fastener may be allowed to translate in situ. A bone fastener may be allowed to translate after at least a portion of the bone fastener has been inserted into a bone segment.

The fixation device and the bone fastener retainer device may be configured to engage each other in a mechanical interlock when the bone fastener retainer device is in its installed position. The mechanical interlock may comprise a tongue-and-groove fit.

Another fixation assembly is described comprising: a fixation device with a plurality of bone fastener openings; and an elastically deflectable bone fastener retainer device configured to be received over the bone fixation device in a mechanical interlock with the bone fixation device.

The fixation device may have opposed edge surfaces separated by a first distance, wherein the bone fastener retainer device has an unstressed condition in which opposite edge surfaces of the bone fastener retainer device are separated by a second distance that is greater than the first distance, and the bone fastener retainer device is elastically deflectable from the unstressed condition to a bowed condition in which the opposite edge surfaces are separated by a third distance that is less than the first distance, such that the opposite edge surfaces of the bone fastener retainer device can be placed between the opposed edge surfaces of the fixation device when the bone fastener retainer device is in the bowed condition, and the bone fastener retainer device can then deflect into the tongue-and-groove fit with the fixation device by rebounding from the bowed condition.

A bone fastener retainer device may be elongated between the opposite edge surfaces. The fixation device may be a spinal fixation plate. A bone fastener openings may be each configured to receive at least a portion of a bone fastener. At least one opening may be circular. At least one opening may be substantially slot-shaped, and have a longitudinal axis. The fixation device may have a pair of circular openings, and a pair of substantially slot-shaped openings.

Another fixation assembly is described comprising: a fixation device with an upper and lower surface, and plurality of bone fastener openings extending from the upper surface to the lower surface; a bone fastener retainer device having an upper and lower surface, and a plurality of bone fastener openings extending from the upper surface to the lower surface; and wherein the bone fastener retainer device is configured to be received over the bone fixation device to prevent bone fastener back-out.

The upper surface of the fixation device may have a first area, and the lower surface of the bone fastener retainer device may have a second area, and wherein the first area and the second area are substantially equal. The fixation device and the bone fastener retainer device may have the same number of bone fastener openings. The location of the openings in the fixation device may generally correspond to the location of the openings in the bone fastener retainer device. The fixation device and the bone fastener retainer device may be configured to be rigidly connected in at least one location.

### BRIEF DESCRIPTION OF THE DRAWINGS

While preferred features of the present invention may be disclosed in the accompanying illustrative, exemplary drawings, for the purposes of description, the invention as defined by the claims should be in no way limited to such preferred features or illustrative and exemplary drawings, wherein:

FIG. 1 is a perspective, exploded view of an embodiment of a fixation apparatus including a bone fastener retainer device;

FIG. 2 is a partial cross-sectional view of a fastener that can be used with the apparatus of FIG. 1;

FIG. 3 is a partial cross-sectional view of fixation plate and retainer device that can be used with the apparatus of FIG. 1;

FIG. 4 is an assembled, partial cross-sectional view of the apparatus of FIG. 1;

FIG. 5 is a perspective, exploded view of another embodiment of a fixation apparatus including a bone fastener retainer device;

FIG. 6 is an assembled cross-sectional view of the fixation apparatus of FIG. 5; and

FIG. 7 is a cross-sectional view of the fixation apparatus of FIG. 5 showing the bending of a retainer device to engage a fixation plate.

### DETAILED DESCRIPTION OF THE INVENTION

The plates described herein may be used in spinal fusion procedures in which a damaged or diseased disc (or part of a disc) is removed from between a pair of vertebrae and a spinal fusion spacer is placed between the vertebrae. The plates may be applied to an anterior portion of the affected vertebrae to span the affected disc space, and may be fixed to the vertebrae using bone screws. The plate may function to maintain the vertebrae aligned during the initial period following fixation in which fusion of the spacer to the adjacent vertebrae occurs. The plate may also function to share some of the axial spinal load applied to the fusion spacer to prevent extreme subsidence of the spacer into the vertebral body, such as where the patient has poor bone quality. The plates may also act to prevent the spacer from being expelled from the disc space during the initial post-operative period.

The plates may be used for single level (i.e. one-disc) or multiple-level (i.e. multiple disc) fusion procedures. Some embodiments may be used for corpectomy procedures, in which at least a portion of a vertebral body is removed. Single level plates generally may have two pairs of bone fastener holes and/or slots, while the multi-level plates generally may have three or more pairs of holes and/or slots. While the plates herein are described with reference and application to the spine, it will be appreciated that features of the plates and the plates may have other applications, and can be applied to other bones and/or parts of the skeleton.

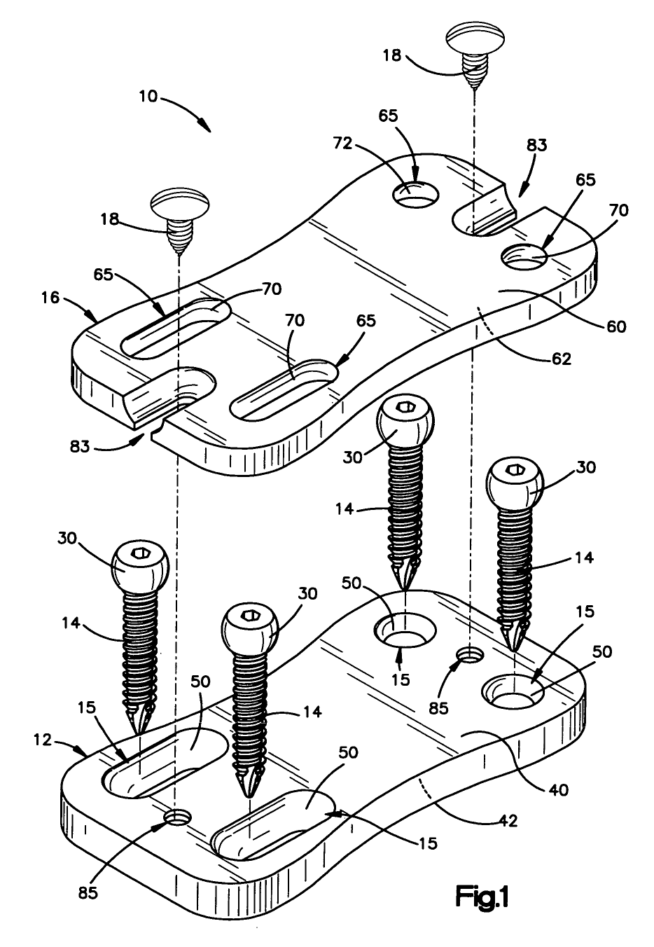

FIGS. 1-4 show an embodiment of a fixation apparatus. The apparatus 10 may include a bone fixation device 12 which, in this particular example, is a spinal fixation plate. A plurality of bone fasteners 14 may be receivable through openings 15 in the fixation plate 12 to fasten it to the spinal column. When the fixation plate 12 and the bone fasteners 14 are fastened to the spinal column, a bone fastener retainer device 16 may be placed over the fixation plate 12 and the bone fasteners 14. A pair of retainer fasteners 18 may then be used to fasten the retainer device 16 to the fixation plate 12 so that the retainer device 16 may block the bone fasteners 14 from backing outward from the fixation plate 12.

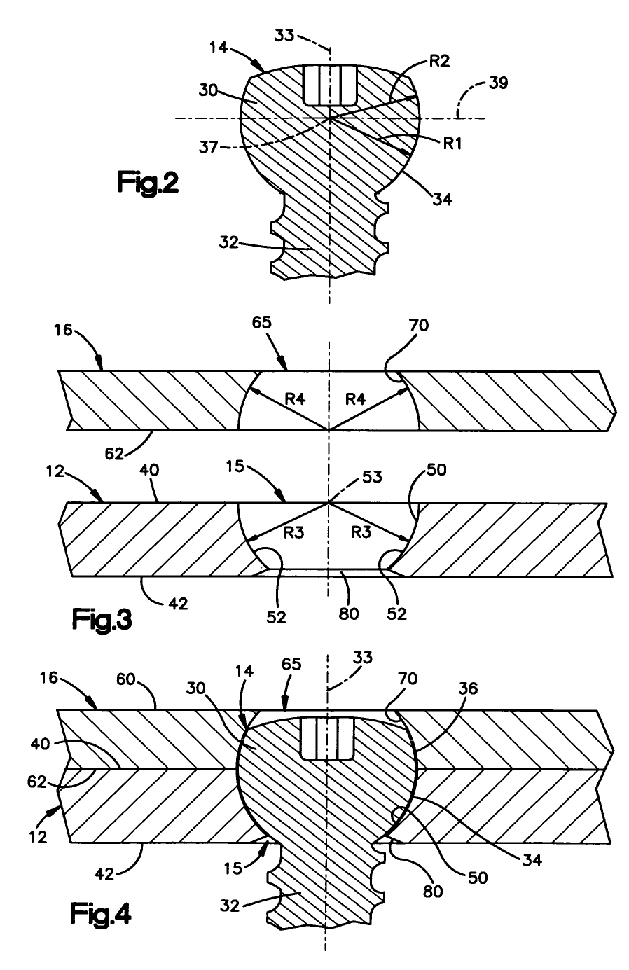

It may be preferable for the bone fasteners 14 to be alike, with each bone fastener 14 having the configuration shown partially in FIG. 2. Each bone fastener 14 thus may have a head 30 and a threaded shank 32 centered on a longitudinal central axis 33. The head 30 may have a convex lower surface 34 and a convex upper surface 36. These two surfaces are shown in FIG. 2 with reference to a point 37 at which the axis 33 intersects a horizontal plane 39. The lower surface 34 may extend downward from the plane 39, and may have an arcuate profile with a radius of curvature R1 extending from the point 37. The convex upper surface 36 may extend upward from the plane 39, and may be smaller than the convex lower surface 34, but also may have an arcuate profile with a radius of curvature R2 extending from the point 37. It may be preferable for the radii of curvature R1 and R2 to be substantially uniform throughout the respective surfaces 34 and 36, and it may also be preferable that they be equal to each other. The two convex surfaces 34 and 36 in this example of the fastener head 30 have the substantially the same spherical contour.

Referring again to FIG. 1, the fixation plate 12 may have a generally rectangular peripheral shape so as to overlie a section of the spine to provide support that maintains the alignment of two or more vertebrae in that section of the spine. The plate 12 is shown in an original condition in which it has planar opposite side surfaces 40 and 42. However, the thickness and material of the plate 12 enable a surgeon to deflect it from a flat configuration as needed for the plate 12 to extend over the spine with an appropriate contour. The thickness and material may be varied to produce a desired amount of deflection depending on the use of the apparatus 10.

The openings 15 in the plate 12 may be arranged in pairs at the opposite end portions of the plate 12. In this arrangement, the pair of bone fasteners 14 at one pair of openings 15 can fasten the plate 12 to a first vertebra, and the pair of bone fasteners 14 at the other pair of openings 15 can fasten the plate 12 to a second vertebra that is spaced from the first vertebra along the length of the spine. Additionally, it may be preferable to have openings 15 in at least one pair are shaped as slots, which may thereby permit the corresponding pair of bone fasteners 14 to move vertically within the slots 15 when compression of the spine causes the two fastened vertebrae to move relatively toward each other lengthwise of the plate 12. It expressly contemplated that the shape and size of each opening 15 in plate 12 may be varied to allow for desired translation of fasteners in response to corresponding vertebral displacement.

Although two of openings 15 shown in FIG. 1 are circular and the other two are slot-shaped, all four of the openings 15 may share the cross-sectional configuration of FIG. 3 when viewed in a direction extending lengthwise of the plate 12. An inner edge surface 50 of the plate 12 may extend around the inside of the opening 15. As viewed in FIG. 3, the inner edge surface 50 may have opposed portions 52 on opposite sides of the opening 15. Those portions 52 of the surface 50 may be provided with contours to match the contour of the lower surface 34 of the screw head 30 (see FIG. 2). Accordingly, in this example, those portions 52 of the surface 50 may each have a uniform radius of curvature R3 extending from a point 53 in the plane of the upper side surface 40, and the radius of curvature R3 may be equal to the radius of curvature R1 at the lower surface 34 of the screw head 30. Alternatively, it may be preferable to dimension radii of curvature R1 and R3 to be slightly offset from one another. Such an arrangement may be beneficial where at least one of the convex lower surface 34 of the screw head 30 and inner edge surface 50 are roughened and/or contoured to provide friction and/or interference between the screw head 30 and the opening 15.

It may be preferable for the retainer plate 16 to have the same peripheral size and shape as the fixation plate 12, as shown in FIG. 1. The retainer plate 16 further may have planar opposite side surfaces 60 and 62, and may be deflectable with the fixation plate 12 when fastened to the fixation plate 12.

As further shown in FIG. 1, the retainer plate 16 may have an array of circular and slot-shaped bone screw openings 65 aligned with the array of openings 15 in the fixation plate 12. As shown in FIG. 3, each of those openings 65 may be bounded by a respective inner edge surface 70, which may be smooth, or may be roughened and/or contoured to provide friction and/or interference for engaging the upper surface 36 of a fastener head 30. Those surfaces 70 may also have concave contours that substantially correspond to the convex contours of the upper surfaces 36 on the fastener heads 30, and the radius of curvature R4 each concave surface 70 may also be substantially equal to the radius of curvature R2 at each convex surfaces 36, or may be alternatively slightly offset. Accordingly, each pair of aligned openings 15 and 65 may be configured to receive a corresponding fastener head 30 in an installed position in which the fastener head 30 is captured between the two plates 12 and 16 in the manner shown in FIG. 4. The convex lower surface 34 of the fastener head 30 may then rest on the concave inner surface 50 of the fixation plate 12 within the opening 15. The concave inner surface 70 of the retainer plate 16 may overlie the convex upper surface 36 of the fastener head 30 within the opening 65 to block the fastener 14 from backing out of the opening 15.

Generally, it may be preferable for the surfaces 50, 70 to correspond to the surfaces 34, 36 of fastener heads 30. However, as discussed above, it may be preferable to offset the shapes and/or dimensions of the surfaces, and/or roughen and/or contour the surfaces, to provide a desired level of friction and/or interference between fastener heads 30 and portions of openings 15. It may also be preferable to vary the shape of surfaces 50, 70 to provide for a relatively looser or tighter fit for a fastener head 30 within an opening 15. For instance, while surfaces 50, 70 may generally be concave, it may be preferable to tilt the surfaces in such a way as to provide a generally convergent or generally divergent opening 15 for a fastener head 30.

While is may generally be preferable for the convex lower surface 34 of a fastener head 30 to be substantially spherical (so that the convex lower surface 34 may correspond to a concave surface 70), it is expressly contemplated that a convex upper surface 36 of a fastener head 30 may be a variety of shapes. For instance, a convex upper surface 36 may be substantially flat, polygonal, oblong, or also spherical.

The fastener heads 30 may be clamped between the two plates 12 and 16 when the retainer fasteners 18 are installed through slots 83 (see FIG. 1) in the retainer plate 16 and further into corresponding threaded bores 85 in the fixation plate 12. In this configuration, the fastener head 30 and the two plates 12 and 16 may together define a ball-and-socket joint for pivotal angulation of the bone fastener 14 relative to the two plates 12 and 16. The range of pivotal angulation of the bone fastener 14 may be defined by an additional inner edge surface 80 (see FIG. 4) of the fixation plate 12 that adjoins the lower side surface 42 at the bottom of the opening 15.

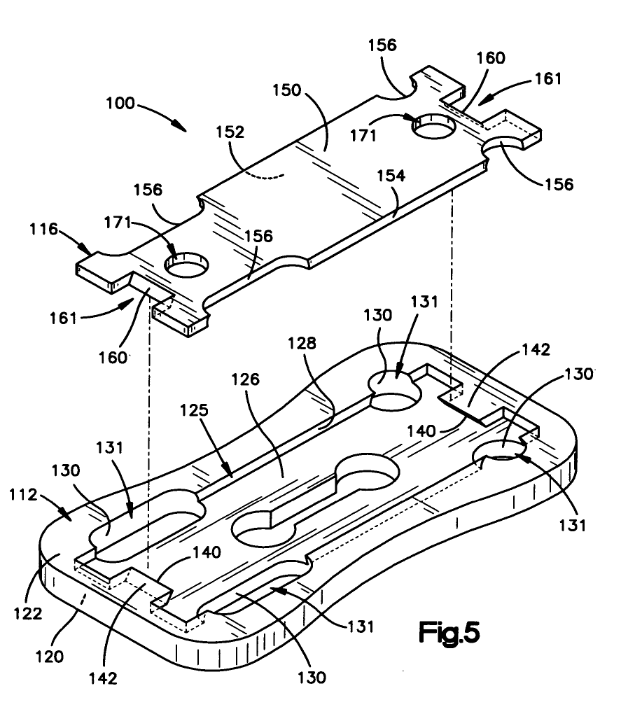

Another embodiment of a fixation apparatus is shown in FIGS. 5-7. The apparatus 100 may include a bone fixation device 110 which, like the bone fixation device 12 described above, is a spinal fixation plate in this particular example. The apparatus 100 likewise may include a bone fastener retainer device 116 in the form of a plate that may be configured to overlie the fixation plate 12 so as to block bone fasteners from backing outward from their installed positions relative to the fixation plate 112.

A lower side surface 120 of the fixation plate 112 may be configured to extend over a section of the spine to which the fixation plate 112 may be fastened by the bone fasteners (not shown). The lower side surface 120, as well as an upper side surface 122, may initially have a planar configuration prior to any deflection of the fixation plate 112 that may occur upon installation on the spine.

The planar upper surface 122 of the fixation plate 112 may surround a compartment 125 configured to receive the retainer plate 116. The bottom of the compartment 125 may be defined by a planar inner surface 126 that may be recessed from the planar upper surface 122. An inner edge surface 128 of the fixation plate 112 may provide the compartment 125 with a uniform depth and a generally rectangular peripheral shape that match the thickness and peripheral shape of the retainer plate 116. Those surfaces 126 and 128 may be interrupted by additional inner edge surfaces 130 that define bone fastener openings 131 at the periphery of the compartment 126.

As shown at the opposite ends of the compartment 125, opposed portions 140 of the inner edge surface 128 may face oppositely toward each other along the length of the compartment 126. Those surfaces 140 may be located at inner ends of tabs 142 that project lengthwise of the compartment 125, and may be inclined downward from the planar upper surface 122 in directions extending oppositely away from each other.

As noted above, the size and shape of the compartment 125 may match the size and shape of the retainer plate 116. More specifically, the retainer plate 116 may have planar upper and lower side surfaces 150 and 152, and also may have a peripheral edge surface 154 that corresponds to the inner edge surface 128 of the fixation plate 112. The peripheral edge surface 154 of the retainer plate 116 may be interrupted by additional peripheral edge surfaces 156. Each of those surfaces 156 may be configured to face across a corresponding bone screw opening 131 in the fixation plate 112 when the retainer plate 116 is installed in the compartment 125. It may be preferable for the peripheral edge surfaces 156 on the retainer plate 116 and the inner edge surfaces 130 on the fixation plate 112 to have contours such that spherical bone fastener heads (not shown) installed in the openings 131 are captured between the two plates 112 and 116 in substantially the same manner that the spherical bone screw heads 30 of FIG. 4 are captured between the two plates 12 and 16.

Additional features of the fixation plate 112 and the retainer plate 116 may enable the two plates to fit together in a mechanical interlock when the retainer plate 116 is installed on the fixation plate 112. This may avoid the use of fasteners, such as the retainer fasteners 18, examples of which are shown in FIG. 1.

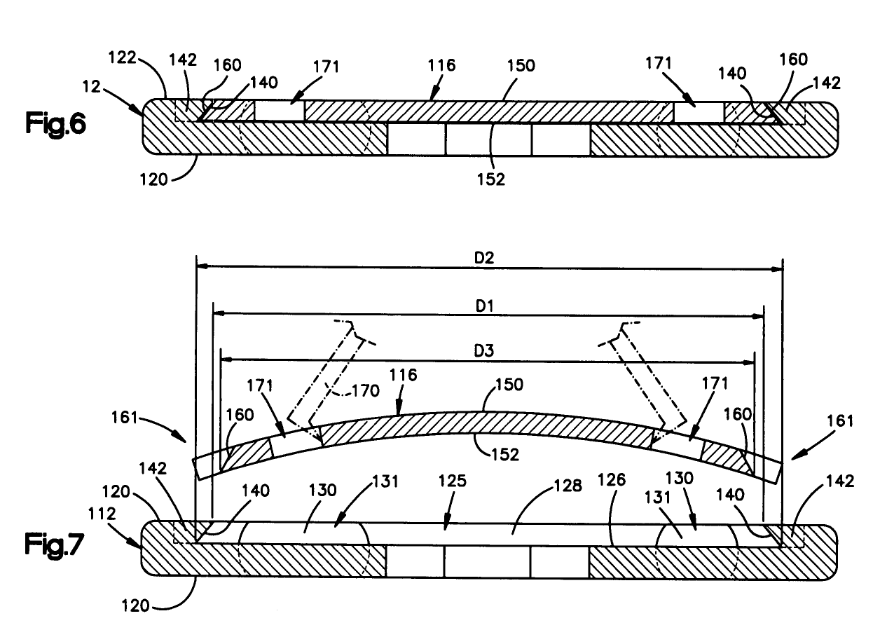

As shown in the illustrated example, the mechanical interlock may be a tongue-and-groove fit. Specifically, portions 160 (see FIG. 5) of the peripheral edge surface 154 may face oppositely away from each other at opposite ends of the retainer plate 116. Those surfaces 160 may be located at the inner ends of a pair of notches 161, and may be inclined oppositely downward and longitudinally outward from the planar upper surface 150. When the retainer plate 16 is installed on the fixation plate 112, as shown in FIG. 6, the tabs 142 on the fixation plate 112 may be received in the notches 161 on the retainer plate 116 such that the oppositely inclined edge surfaces 140 and 160 may establish an tongue-and-groove fit between the two plates 112 and 116.

The retainer plate 116 can be placed in its installed position by first deflecting it elastically from the original, unstressed condition of FIG. 5 to the bowed condition of FIG. 7. This may be necessary because the distance D1 between the opposed edge surfaces 140 on the fixation plate 112 may be less than the original distance D2 between the opposite edge surfaces 160 on the retainer plate 116. When the retainer plate 116 has been deflected into a bowed condition in which the distance between the opposite edge surfaces 160 is reduced to a distance D3 that is less than the distance D1, it can be moved downward between the opposed edge surfaces 140, and can then move toward and into its installed position upon rebounding from the bowed condition. Installation and removal of the retainer plate 116 can be accomplished in this manner by the use of a scissor-action surgical tool 170, shown schematically in phantom view in FIG. 7, that may engage the retainer plate 116 in apertures 171 provided for that purpose.

Retainer plate 116 may be comprised of a generally flexible material, such as timoly (titanium alloy with approximately 15% molybdenum).

It should be noted that the aforementioned descriptions and illustrations have been provided as examples of the configurations of translation plates that may be designed and assembled using the principles of the invention. These examples will be understood to one of ordinary skill in the art as being non-limiting in that a translating plate employing one or more of the disclosed features may be produced as desired or required for a particular patient's need. Thus, the features disclosed are “modular” in nature.

Each of the fasteners and fixation plates disclosed herein may be formed of a titanium alloy such as titanium-aluminum-niobium, which may be anodized. One material for use with each of the plates and screws described herein is Ti-6Al-7Nb, with a density of about 4.52 gm/cc, a modulus of elasticity of about 105 GPa, an ultimate tensile strength of about 900 MPa, and a yield strength of about 800 MPa. Surfaces of the fasteners may also be burr free, with all sharp edges having a radius to a maximum of about 0.1 mm.

While the invention has been shown and described herein with reference to particular embodiments, it is to be understood that the various additions, substitutions, or modifications of form, structure, arrangement, proportions, materials, and components and otherwise, used in the practice and which are particularly adapted to specific environments and operative requirements, may be made to the described embodiments without departing from the spirit and scope of the present invention. Accordingly, it should be understood that the embodiments disclosed herein are merely illustrative of the principles of the invention. Various other modifications may be made by those skilled in the art which will embody the principles of the invention and fall within the spirit and the scope thereof.

This written description sets forth the best mode of the claimed invention, and describes the claimed invention to enable a person of ordinary skill in the art to make and use it, by presenting examples of the elements recited in the claims. The patentable scope of the invention is defined by the claims themselves, and may include other examples that occur to those skilled in the art. Such other examples, which may be available either before or after the application filing date, are intended to be within the scope of the claims if they have structural elements that do not differ from the literal language of the claims, or if they include equivalent structural elements with insubstantial differences from the literal language of the claims.

1. A fixation assembly comprising:
a bone fastener having a head, the head having a convex lower surface with a first arcuate contour and a convex upper surface with a second arcuate contour;
a fixation device configured to receive the bone fastener in an installed position, wherein the convex lower surface of the fastener head rests on a concave surface of the fixation device also substantially having the first arcuate contour; and
a bone fastener retainer device configured to be mounted on the fixation device in an installed position, wherein a concave surface of the bone fastener retainer device overlies the convex upper surface of the head, wherein the concave surface of the bone fastener retainer device also substantially has the second arcuate contour.

2. The assembly of claim 1, wherein the first and second arcuate contours are substantially spherical.

3. The assembly of claim 2, wherein the first and second arcuate contours have substantially equal radii of curvature.

4. The assembly of claim 1, wherein the fixation device is a plate, and wherein the concave surface of the fixation device is an inner edge surface within an opening through the plate.

5. The assembly of claim 4, wherein the opening is substantially slot-shaped, and capable of receiving at least a portion of a bone fastener, the opening further having a longitudinal axis.

6. The assembly of claim 5, wherein the bone fastener is allowed to translate within the opening along the longitudinal axis.

7. The assembly of claim 6, wherein the bone fastener is allowed to translate in situ.

8. The assembly of claim 6, wherein the bone fastener is allowed to translate after at least a portion of the bone fastener has been inserted into a bone segment.

9. The assembly of claim 1, wherein the fixation device and the bone fastener retainer device are configured to engage each other in a mechanical interlock when the bone fastener retainer device is in its installed position.

10. The assembly of claim 9, wherein the mechanical interlock comprises a tongue-and-groove fit.

11. A fixation assembly comprising:
a fixation device with a plurality of bone fastener openings; and
an elastically deflectable bone fastener retainer device configured to be received over the bone fixation device in a mechanical interlock with the bone fixation device.

12. The assembly of claim 11, wherein the mechanical interlock comprises a tongue-and-groove fit.

13. The assembly of claim 12, wherein the fixation device has opposed edge surfaces separated by a first distance, wherein the bone fastener retainer device has an unstressed condition in which opposite edge surfaces of the bone fastener retainer device are separated by a second distance that is greater than the first distance, and the bone fastener retainer device is elastically deflectable from the unstressed condition to a bowed condition in which the opposite edge surfaces are separated by a third distance that is less than the first distance, such that the opposite edge surfaces of the bone fastener retainer device can be placed between the opposed edge surfaces of the fixation device when the bone fastener retainer device is in the bowed condition, and the bone fastener retainer device can then deflect into the tongue-and-groove fit with the fixation device by rebounding from the bowed condition.

14. The assembly of claim 13, wherein the bone fastener retainer device is elongated between the opposite edge surfaces.

15. The assembly of claim 13, wherein the fixation device is a spinal fixation plate.

16. The assembly of claim 11, wherein the bone fastener openings are each configured to receive at least a portion of a bone fastener.

17. The assembly of claim 16, wherein at least one opening is circular.

18. The assembly of claim 16, wherein at least one opening is substantially slot-shaped, and has a longitudinal axis.

19. The assembly of claim 18, wherein a bone fastener is allowed to translate along the longitudinal axis of the opening.

20. The assembly of claim 19, wherein the bone fastener is allowed to translate in situ.

21. The assembly of claim 19, wherein the bone fastener is allowed to translate after at least a portion of it is inserted into a bone segment.

22. The assembly of claim 11, wherein the fixation device has a pair of circular openings, and a pair of substantially slot-shaped openings.

23. A fixation assembly comprising:
a fixation device with an upper and lower surface, and plurality of bone fastener openings extending from the upper surface to the lower surface;
a bone fastener retainer device having an upper and lower surface, and a plurality of bone fastener openings extending from the upper surface to the lower surface; and
wherein the bone fastener retainer device is configured to be received over the bone fixation device to prevent bone fastener back-out.

24. The assembly of claim 23, wherein the upper surface of the fixation device has a first area, and the lower surface of the bone fastener retainer device has a second area, and wherein the first area and the second area are substantially equal.

25. The assembly of claim 23, wherein the fixation device and the bone fastener retainer device have the same number of bone fastener openings.

26. The assembly of claim 25, wherein the location of the openings in the fixation device generally correspond to the location of the openings in the bone fastener retainer device.

27. The assembly of claim 23, wherein at least one opening is circular.

28. The assembly of claim 23, wherein at least one opening is substantially slot-shaped, and has a longitudinal axis.

29. The assembly of claim 23, wherein a bone fastener is allowed to translate along the longitudinal axis of the opening.

30. The assembly of claim 29, wherein the bone fastener is allowed to translate in situ.

31. The assembly of claim 29, wherein the bone fastener is allowed to translate after at least a portion of it is inserted into a bone segment.

32. The assembly of claim 23, wherein the fixation device has a pair of circular openings, and a pair of substantially slot-shaped openings.

33. The assembly of claim 23, wherein the fixation device and the bone fastener retainer device are configured to be rigidly connected in at least one location.
